(19) United States
(12) Patent Application Publication
(10) Pub. No.: US 2023/0103891 A1
(43) Pub. Date: Apr. 6, 2023

(54) DRUG-LOADED NANOPARTICLES FOR HEPATIC ARTERY CHEMOEMBOLIZATION AND PREPARATION METHOD THEREOF

(71) Applicant: The First Affiliated Hospital of Sun Yat-Sen University, Guangzhou (CN)

(72) Inventors: Jiaping Li, Guangzhou (CN); Lingwei Liu, Guangzhou (CN)

(73) Assignee: The First Affiliated Hospital of Sun Yat-Sen University, Guangzhou (CN)

(21) Appl. No.: 18/074,561

(22) Filed: Dec. 5, 2022

(30) Foreign Application Priority Data
Feb. 22, 2022 (CN) 202210163251.6

(51) Int. Cl. A61L 24/00

(52) U.S. Cl. CPC A61L 24/0015; A61L 24/0068; A61L 2400/12; A61L 2430/36; A61L 2300/232

(57) ABSTRACT

The present disclosure describes drug-loaded nanoparticles for hepatic artery chemoembolization, wherein the drug-loaded nanoparticles are novel doxorubicin-loaded metal organic framework (MOF) nanoparticles and UiO-66/Bi₂S₃ nanocomposites. The preparation method of the drug-loaded nanoparticles includes the following steps: mixing UiO-66/Bi₂S₃ with DOX solution, stirring the resultant mixture overnight at 25° C.±5° C. in a dark environment, centrifuging the mixture, and washing with deionized water to obtain UiO-66/UiO-66/Bi₂S₃@DOX composite nanomaterials. The present disclosure adopts a “one-pot reaction” with a simple preparation process. The pH-reactive release performance of the MOF material indicates that the tumor tissue of hepatocellular carcinoma (HCC) has a lower pH than normal tissue, and the acidic tumor environment can induce the release of doxorubicin from the nanomaterials. The MOF material has strong photothermal conversion ability, allowing HCC to be treated by photothermal treatment in combination with TACE.

### TECHNICAL FIELD

The present disclosure relates to the technical solution of hepatic artery chemoembolization, particularly to drug-loaded nanoparticles for hepatic artery chemoembolization and a preparation method thereof.

### BACKGROUND

Currently, drug-eluting bed (DEB) TACE (DEB-TACE) is widely used in clinical TACE treatment. DEBs are a novel embolic material that can adsorb and carry chemotherapy drugs and are not biodegradable in vivo. After entering tumor blood vessels, DEBs can embolize the tumor blood vessels for a prolonged period and allow the chemotherapy drugs to act on the tumor interior for 7-14 days. These two effects can be combined to achieve better local control.

Owing to the limited material properties, the existing DEB drug-loaded microspheres do not exhibit pH-reactive release and photothermal conversion ability. For example, a Chinese patent document, CN109789226A, discloses a composition for hepatic arterial chemoembolization, including an embolic material, and human serum albumin nanoparticles carrying a water-soluble anticancer agent; and a method for preparing the composition. The method includes dispersing human serum albumin nanoparticles carrying a water-soluble anticancer agent in a computed tomography and X-ray contrast medium and mixing the dispersed nanoparticles with an embolic material, wherein the mixture of the nanoparticles and the microbubbles is mixed with the embolic material. The drug-loaded nanoparticles described in the document are different from the product technology of the present disclosure, and the prepared product has low pH-reactive release and photothermal conversion ability and high process complexity.

Therefore, the present disclosure provides drug-loaded nanoparticles for hepatic artery chemoembolization and the preparation method thereof.

### SUMMARY

In view of the defects of the prior art, an object of the present disclosure is to provide drug-loaded nanoparticles with a simple preparation process, pH-reactive release, and photothermal conversion ability for hepatic artery chemoembolization.

To solve the technical problems, the present disclosure provides the following technical solutions:

The present disclosure provides drug-loaded nanoparticles for hepatic artery chemoembolization, wherein the drug-loaded nanoparticles are novel doxorubicin (DOX)-loaded metal organic framework (MOF) nanoparticles.

Preferably, the nanoparticles are UiO-66/UiO-66/Bi₂S₃ nanocomposites.

The present disclosure provides a preparation method for the drug-loaded nanoparticles, comprising the following steps:

Step 1: dissolving 0.0772 g of bismuth acetate (Bi(C₂H₃O₂)₃) in 30 mL of ethylene glycol to form a homogeneous solution, dissolving 0.175 g of UiO-66-NH₂ in 5 mL of ethylene glycol under ultrasonic stirring for 25 to 35 min, followed by the addition of Bi(C₂H₃O₂)₃ and slow stirring at 25° C.±5° C. for 55 to 65 min;

Step 2: adding Na₂S solution dropwise, continue stirring for 25 to 35 min, then transferring the suspension to a stainless-steel hydrothermal reactor, and letting it stand at 85 to 95° C. for 0.8 to 1.2 h followed by centrifugation and washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging and washing it with deionized water to obtain UiO-66/UiO-66/Bi₂S₃@DOX composite nanomaterials. Preferably, the Na₂S solution is added in an amount of 1 mL, and the Na₂S solution has a mass concentration of 0.3 mol/L.

Preferably, the stainless-steel hydrothermal reactor has a volume of 100 mL.

Preferably, UiO-66/UiO-66/Bi₂S₃ has a concentration of 0.1 to 0.9 mg/mL.

Preferably, UiO-66/UiO-66/Bi₂S₃ has a concentration of 0.5 mg/mL.

Preferably, the DOX solution has a concentration of 0.8 to 1.2 mg/mL.

Preferably, the DOX solution has a concentration of 1.0 mg/mL.

Compared with the prior art, the present disclosure has several advantages. First, the present disclosure adopts a “one-pot reaction” with a simple preparation process. Additionally, the pH-reactive release performance of the MOF material indicates that the tumor tissue of hepatocellular carcinoma (HCC) has a lower pH than that of normal tissue, and the acidic tumor environment can induce the release of DOX from the nanomaterials. Finally, the MOF material has strong photothermal conversion ability, allowing HCC to be treated by photothermal ablation in combination with TACE.

### BRIEF DESCRIPTION OF THE DRAWINGS

FIG. 1 shows an electron microscope image of drug-loaded nanoparticles for hepatic artery chemoembolization according to the present disclosure.

FIG. 2 shows a schematic diagram of the pH-reactive release of the drug-loaded nanoparticle materials for hepatic artery chemoembolization according to the present disclosure.

FIG. 3 shows a schematic diagram of photothermal performance of the drug-loaded nanoparticle materials for hepatic artery chemoembolization according to the present disclosure.

FIG. 4 is a diagram showing therapeutic effects of animal experiments of the drug-loaded nanoparticle materials for hepatic artery chemoembolization according to the present disclosure.

### DETAILED DESCRIPTION OF THE EMBODIMENTS

The technical solutions in the examples of the present disclosure are clearly and completely described below. Obviously, the examples described are only some of the examples of the present disclosure. All other examples that persons of ordinary skill in the art obtain without creative efforts based on the examples of the present disclosure also fall within the scope of the present disclosure.

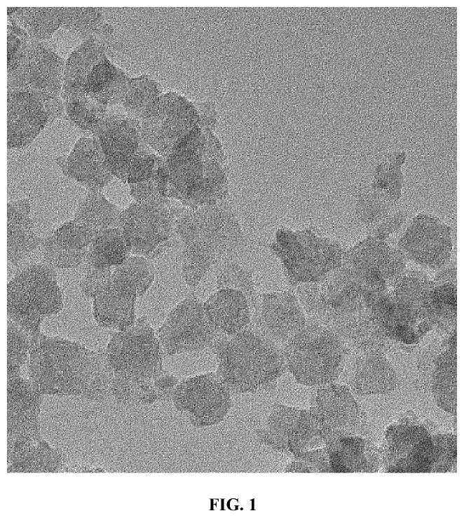

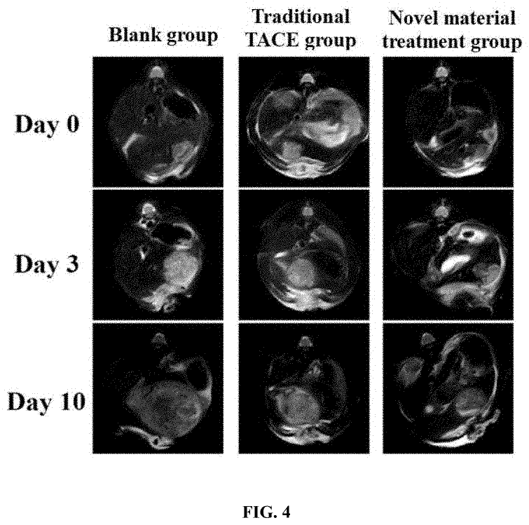

Referring to FIGS. 1-4,

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 25 to 35 min, followed by the addition of Bi(C₂H₃O₂)₃ and slow stirring at 25° C.±5° C. for 55 to 65 min;

Step 2: adding Na₂S solution dropwise, continuing stirring for 25 to 35 min, then transferring the suspension to a stainless-steel hydrothermal reactor, letting it stand at 85 to 95° C. for 0.8 to 1.2 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.1 to 0.9 mg/mL.

In this example, the DOX solution has concentration of 0.8 to 1.2 mg/mL.

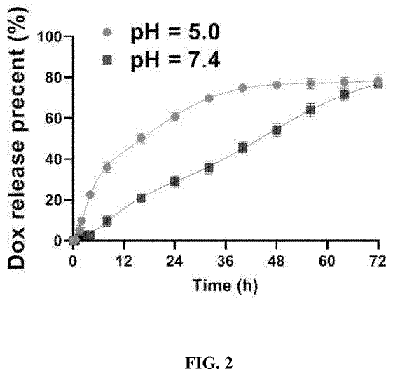

FIG. 2 shows the in vitro pH-reactive release of DOX: 25 mg of UiO-66/UiO-66/Bi₂S₃@Dox were immersed in 10 mL of phosphate buffered saline (PBS) at two different pH values (7.4 and 5.0), shaken, and mixed at 37° C. to conduct a DOX release experiment in vitro. Then, 5 mL of the solution was removed again and replaced with an equal volume of fresh PBS solution with the same pH value.

Using an ultraviolet-visible spectrophotometer, the content of DOX molecules released at different pH values was calculated at a wavelength below 478 n, and the percentage of DOX released was calculated as follows: release percentage (cumulative amount of DOX released each time)/(total amount of DOX loaded on UiO-66/UiO-66/Bi₂S₃)×100%. Three tests were conducted on each sample.

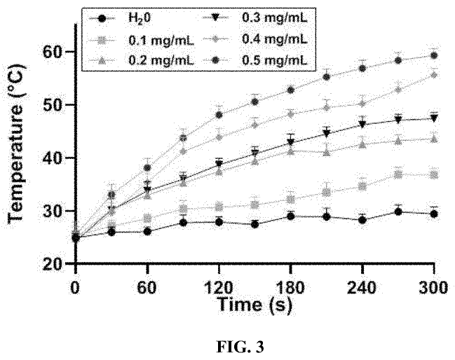

In FIG. 3, a near-infrared laser was used as a fiber-coupled laser light source (MDL-H-808-5W) in vitro to detect UiO-66/Bi₂S₃ nanoparticles with concentrations of 0 mg/mL (water), 0.1 mg/mL, 0.2 mg/mL, 0.3 mg/mL, 0.4 mg/mL, and 0.5 mg/mL in EP tubes with a wavelength of 808 nm, and a power density of 2.0 W/cm², followed by irradiation at 25° C.±5° C. for 5 min. An infrared thermal imager (FLIR) was used to monitor the temperature change, record the indoor temperature, and draw temperature profiles.

### (I) Example 1

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 25 min, followed by the addition of Bi(C₂H₃O₂)₃ and slow stirring at 25° C.±5° C. for 55 min;

Step 2: adding Na₂S solution dropwise, continuing stirring for 25 min, then transferring the suspension to a stainless-steel hydrothermal reactor, letting it stand at 85° C. for 0.8 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.1 mg/mL.

In this example, the DOX solution has a concentration of 0.8 mg/mL.

### (II) Example 2

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 35 min, followed by the addition of Bi(C₂H₃O₂)₃ and slow stirring at 25° C.±5° C. for 65 min.

Step 2: adding Na₂S solution dropwise, continuing stirring for 35 min, then transferring the suspension to a stainless-steel hydrothermal reactor, letting it stand at 95° C. for 1.2 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.9 mg/mL.

In this example, the DOX solution has a concentration of 1.2 mg/mL.

### (III) Example 3

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 30 min, followed by the addition of Bi(C₂H₃O₂)₃ and slow stirring at 25° C.±5° C. for 60 min;

Step 2: adding Na₂S solution dropwise, continuing stirring for 30 min, and transferring the suspension to a stainless-steel hydrothermal reactor, letting it stand at 90° C. for 1.0 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.5 mg/mL.

In this example, the DOX solution has a concentration of 1.0 mg/mL.

### (IV) Example 4

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 27 min, followed by the addition of Bi(C₂H₃O₂)₃ and slow stirring at 25° C.±5° C. for 55 to 65 min;

Step 2: adding Na₂S solution dropwise, continuing stirring for 26 min, then transfer the suspension to a stainless-steel hydrothermal reactor, letting it stand at 86° C. for 0.9 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.1 mg/mL.

In this example, the DOX solution has a concentration of 0.9 mg/mL.

### (V) Example 5

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution, dissolving 0.175 g of UiO-66-NH₂ in 5 mL of ethylene glycol under ultrasonic stirring for 25 to 35 min, followed by the addition of Bi(C₂H₃O₂)₃ and slow stirring at 25° C.±5° C. for 62 min;

Step 2: adding Na₂S solution dropwise, continuing stirring for 33 min, then transfer the suspension to a stainless-steel hydrothermal reactor, letting it stand at 92° C. for 1.1 h followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.8 mg/mL.

In this example, the DOX solution has a concentration of 1.1 mg/mL.

### (I) Comparative Example 1

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution. Dissolve 0.175 g of UiO-66-NH₂ in 5 mL of ethylene glycol under ultrasonic stirring for 20 min, followed by the addition of Bi(C₂H₃O₂)₃ and slow stirring at 25° C.±5° C. for 50 min;

Step 2: adding Na₂S solution dropwise, continuing stirring for 20 min, then transferring the suspension to a stainless-steel hydrothermal reactor, letting it stand at 80° C. for 0.6 h, followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 0.08 mg/mL.

In this example, the DOX solution has a concentration of 0.6 mg/mL.

### (II) Comparative Example 2

This example describes drug-loaded nanoparticles for hepatic artery chemoembolization, wherein the drug-loaded nanoparticles are novel DOX-loaded MOF nanoparticles and UiO-66/UiO-66/Bi₂S₃ nanocomposites.

This example provides a preparation method for drug-loaded nanoparticles, comprising the following steps:

Step 1: dissolving 0.0772 g of Bi(C₂H₃O₂)₃ in 30 mL of ethylene glycol to form a homogeneous solution. dissolving 0.175 g of UiO-66-NH₂ in 5 mL of ethylene glycol under ultrasonic stirring for 38 min, followed by the addition of Bi(C₂H₃O₂)₃ and slow stirring at 25° C.±5° C. for 70 min;

Step 2: adding Na₂S solution dropwise, continuing stirring for 40 min, then transferring the suspension to a stainless-steel hydrothermal reactor, letting it stand at 98° C. for 1.3 h followed by centrifugation, washing it with ethanol and deionized water, and drying it in an oven at 80° C. for 24 h to obtain UiO-66/Bi₂S₃; and

Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

In this example, the Na₂S solution is added in an amount of 1 mL, and the Na₂S solution has a mass concentration of 0.3 mol/L.

In this example, the stainless-steel hydrothermal reactor has a volume of 100 mL.

In this example, UiO-66/UiO-66/Bi₂S₃ has a concentration of 1.0 mg/mL.

In this example, the DOX solution has a concentration of 1.3 mg/mL.

For those skilled in the art, it is obvious that the present disclosure is not limited to the details of the above examples and can be implemented in other specific forms without departing from the spirit or basic features of the present disclosure. Thus, the examples should be regarded as exemplary and nonlimiting. The scope of the present disclosure is defined by appended claims rather than by the above descriptions. Therefore, it is intended to include all variations within the meaning and range of the equivalent elements of claims in the present disclosure.

In addition, it should be understood that although the description is written according to the examples, not every example contains only an independent technical solution. This description is provided only for the sake of clarity. Those skilled in art should take the description as a whole, and the technical solutions in each example can also be properly combined to form other examples that can be understood by those skilled in art.

What is claimed is:

1. Drug-loaded nanoparticles for hepatic artery chemoembolization, wherein the drug-loaded nanoparticles are novel doxorubicin (DOX)-loaded metal organic framework (MOF) nanoparticles.

2. A preparation method for the drug-loaded nanoparticles according to claim 1, wherein the nanoparticles are UiO-66/Bi₂S₃ nanocomposites.

3. The preparation method for the drug-loaded nanoparticles according to claim 1 comprising of the following steps:
Step 1: dissolving 0.0772 g of bismuth acetate (Bi(C₂H₃O₂)₃) in 30 mL of ethylene glycol to form a homogeneous solution, dissolving 0.175 g of UiO-66-NH₂ in 5 mL of ethylene glycol under ultrasonic stirring for 25 to 35 min, followed by the addition of Bi(C₂H₃O₂)₃, and stir at 25° C.±5° C. for 55 to 65 min;
Step 2: adding Na₂S solution dropwise, and continuing stirring for 25 to 35 min, then transferring the suspension to a stainless-steel hydrothermal reactor, and letting it stand at 85 to 95° C. for 0.8 to 1.2 h, followed by centrifugation, next, washing it with ethanol and deionized water, and drying it in an oven at 80° C. for 24 h to obtain UiO-66/Bi₂S₃; and
Step 3: mixing the obtained UiO-66/Bi₂S₃ with the DOX solution, stirring the resultant mixture overnight at 25° C.±5° C. in a dark environment, centrifuging, and washing it with deionized water to obtain UiO-66/UiO-66/Bi₂S₃@DOX composite nanomaterials.

4. The preparation method for the drug-loaded nanoparticles according to claim 3, wherein 1 mL of Na₂S solution is added, and the Na₂S solution has a mass concentration of 0.3 mol/L.

5. The preparation method for the drug-loaded nanoparticles according to claim 3, wherein the stainless-steel hydrothermal reactor has a volume of 100 mL.

6. The preparation method for the drug-loaded nanoparticles according to claim 3, wherein UiO-66/UiO-66/Bi₂S₃ has a concentration of 0.1 to 0.9 mg/mL.

7. The preparation method for the drug-loaded nanoparticles according to claim 6, wherein UiO-66/UiO-66/Bi₂S₃ has a concentration of 0.1 to 0.9 mg/mL.

8. The preparation method for the drug-loaded nanoparticles according to claim 3, wherein the DOX solution has a concentration of 0.8 to 1.2 mg/mL.

9. The preparation method for the drug-loaded nanoparticles according to claim 8, wherein the DOX solution has a concentration of 1.0 mg/mL.
